(19) United States
(12) Patent Application Publication
(10) Pub. No.: US 2013/0090449 A1
(43) Pub. Date: Apr. 11, 2013

(54) Biobased Polyester Diols And Polyols From High Molecular Weight Polyhydroxyalkanoates

(71) Applicant: Metabolix, Inc., Cambridge, MA (US)

(72) Inventor: Robert S. Whitehouse, Cambridge, MA (US)

(73) Assignee: METABOLIX, INC., Cambridge, MA (US)

(21) Appl. No.: 13/628,099

(22) Filed: Sep. 27, 2012

Related U.S. Application Data
(60) Provisional application No. 61/543,584, filed on Oct. 5, 2011.

(51) Int. Cl. C07C 69/66; C08G 18/10

(52) U.S. Cl. 528/60; 560/182

(57) ABSTRACT

Methods for producing biobased hydroxyl-terminated polyester polymers are described wherein molten-dispersed or hydrocarbonaceous fluid-dispersed phase polyhydroxyalkanoate polymers are reacted with a difunctional hydroxylated insertion compound at a sufficient temperature and time to depolymerize the starting polyester polymers to provide hydroxyl-terminated reaction products of reduced molecular weight. The difunctional hydroxylated insertion compound can be at least one of diol, aminoalcohol, and polyhydroxyalcohol. The use of catalyst may be required, reduced or eliminated. The hydroxyl-terminated reaction product is at least one of hydroxyl-terminated polyhydroxyalkanoate. A method for making polyurethanes further is described wherein the hydroxyl-terminated reaction product is reacted with polyfunctional isocyanate to form a polyurethane product. Products that include the hydroxyl-terminated reaction product or polyurethane also are described.

This application claims the benefit under 35 U.S.C. §119(e) of prior U.S. Provisional Patent Application No. 61/543,584, filed Oct. 5, 2011, which is incorporated in its entirety by reference herein.

### BACKGROUND OF THE INVENTION

The present invention relates to methods for making biobased polyester diols and polyols from polyhydroxyalkanoates as well as polyurethane products made from them. More specifically, the present invention relates to polyester diols and polyols derived in part from high molecular weight biobased polyhydroxyalkanoate homopolymers, copolymers, and blends of one or more homopolymer or copolymer. Other aspects of the present invention are provided below.

In general, the raw materials for preparing polyurethanes include polyisocyanates, polyols, amines, catalysts, additives, optional foaming agents, and the like. The polyisocyanates are either aliphatic, like hexamethylene diisocyanates, isophorone diisocyanate, and 4,4′-diisocyanate dicyclo hexylmethane, or the polyisocyanates can be aromatic, like 2,4-toluene diisocyanate, 1,5-naphthalene diisocyanate, and 4,4′-methylene diphenyl diisocyanate. The polyols are typically polyethers, such as propylene glycol and trimethylolpropane combined with sucrose or polyesters, or ethylene glycol, 1,2-propanediol, 1,4-butenediol, and diethylene glycol combined with glycerol. Polyether polyols are typically used to produce flexible and rigid foams while polyester polyols are typically used to produce elastomers, flexible foams, and coatings.

The polyester type polyols are normally prepared by reaction of petroleum-based dialcohols with dicarboxylic acids, which typically involve chemically building the desired molecules with smaller molecules. However with the dwindling petroleum resources, increasing energy prices, and environmental concerns, there has been significant interest in the development of large scale processes to produce chemicals from biobased, renewable resources. One class of materials that could be used to develop new biobased polyester polyols for formulating polyurethane materials are polyhydroxyalkanoates.

Biobased polyester polymers such as polyhydroxybutyrates (PHB's) are naturally produced in biomass systems, such as plant biomass, microbial biomass (e.g., bacteria including cyanobacteria, yeast, fungi) or algae biomass. Genetically-modified biomass systems have recently been developed which produce a wide variety of biodegradable PHB polymers and copolymers (Lee (1996), Biotechnology & Bioengineering 49:1-14; Braunegg et al. (1998), J. Biotechnology 65:127-161; Madison, L. L. and Huisman, G. W. (1999), Metabolic Engineering of Poly-3-Hydroxyalkanoates; From DNA to Plastic, in: Microbiol. Mol. Biol. Rev. 63:21-53). Polylactic acid (PLA) is another biobased polyester polymer that is produced from renewable resources like corn sugar and comes in different structural forms (poly-L-lactide or poly-D-lactide) depending on the chirality of the lactic acid used to create the polymer. A range of physical properties can be achieved simply by combining the D and L forms of PLA together. Both the polyhydroxybutyrates and polylactic acids belong to a general class of biodegradable materials called polyhydroxyalkanoates.

It has been demonstrated that PHA's can be used as starting materials for the production of biobased chemicals used in polyurethane formulations (e.g., U.S. Pat. No. 7,230,144). However, the present investigator has realized that there is a need to develop new biobased polyester polyols coupled with new processes that are simple, energy efficient, flexible and scalable in order to produce quantities that are commensurate with a world-wide polyurethane market that is estimated to be in the billions of dollars.

### SUMMARY OF THE PRESENT INVENTION

A feature of the present invention is to provide biobased polyester diols and polyols that are derived in part by a controlled/systematic depolymerization of higher molecular weight biobased polyhydroxyalkanoate (“PHA”) polymers and copolymers with or without a catalyst present.

A further feature of the present invention is to provide biobased polyester diols and polyols that are derived in part from controlled/systematic chain breaking reactions performed with an insertion compound on higher molecular weight biobased PHA polymers and copolymers.

A further feature of the present invention is to provide biobased polyester diols and polyols that are derived in part by a controlled/systematic depolymerization of higher molecular weight biobased PHA's, wherein a repeat unit of the original PHA can be essentially maintained throughout the resulting polymer chain fragments other than at end-chain groups of the polyester diol and polyol products.

A further feature of the present invention is to provide biobased polyester diols and polyols that are derived in part by a controlled/systematic depolymerization of higher molecular weight biobased PHA's, wherein hydroxyl groups can be attached on both ends of the polyester diol or polyol products or alternatively, ester groups in the starting PHA's can be selectively cleaved to produce a hydroxyalkanoate ester on one side of the product polymer chain and a hydroxyl functional group attached to the alkanoate acid group on the other side of the same product polymer chain. For example, poly-3-hydroxybutyrate may be reacted with ethanolamine as the insertion molecule to provide a poly-3-hydroxybutyrate chain with a secondary hydroxyl group at one end and an ethanolamide linkage through the terminal carboxylic acid, providing a polymer with primary and secondary functionality. Similarly, if diethanolamine is used in preference to ethanolamine then a diethanolamide is the resulting terminal group, and in this case the PHA polyols have two primary hydroxyl groups and one secondary hydroxyl groups thus making it useful in branched polyurethane compositions. Further, if propylene glycol is used as a replacement for ethanolamine, then the primary hydroxyl group on the 1,2-propanediol insert into the butyrate ester and the resulting polyol now has statistically two secondary hydroxyl groups whilst if 1,3-propanediol or butanol are used, the PHA polyols have a primary and secondary terminal hydroxyl groups. Further, if glycerol is used as the insertion molecule, then a trihydroxyl PHA polyol with one primary hydroxyl and two secondary hydroxyl groups is produced.

A further feature of the present invention is to provide biobased polyester diols and polyols that are derived in part by a controlled/systematic depolymerization of higher molecular weight biobased PHA's to yield smaller molecular weight polyester diol and polyol products which can have high molecular weights, such as up to about 200,000, and which have end caps that provide a pair of functional groups through which these polymer products can provide reactive sites for further reactions useful in the manufacture of PHA derivatives, such as polyurethanes or other materials.

A further feature of the present invention is to provide biobased polyester diols and polyols which are PHA diols and polyols as derived in part from biobased higher molecular weight PHA polymers and copolymers by a melt-phase based transesterification method or a hydrocarbonaceous fluid (nonaqueous) dispersion-phase based transesterification method, which can increase conversion rates, reduce unwanted side reactions, reduce catalyst requirements, improve “dial-in” capabilities on product molecular structures and molecular weights, improve product purity, or provide other benefits and advantages.

A further feature of the present invention is to provide biobased polyester diols and polyols which are PHA diols and polyols as derived in part from biobased higher molecular weight PHA polymers and copolymers by a hydrocarbonaceous dispersion-phase based transesterification method which can use synergistic blends of different nonsolvents for solution dispersion, which blend components individually have no significant solvent effects on the PHA and/or normally require a catalyst to interact with PHA, that together increase solubility of the reaction mix to accelerate the reaction conversion as compared with a heterophase degradation reaction.

A further feature of the present invention is to provide biobased polyester diols and polyols which are PHA diols and polyols as derived in part from biobased higher molecular weight PHA polymers and copolymers in a transesterification method with reduced or eliminated use of catalyst, wherein catalyst removal and recovery steps can be eliminated during processing and catalyst-free products can be more easily provided.

A further feature of the present invention is to provide polyurethanes derived from the biobased, polyester diols and polyols.

A further feature of the present invention is to provide products, such as polyhydroxyalkanoates, compositions and articles, directly containing or formed with the biobased polyester diols and polyols, or polyurethanes derived therefrom, or both.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates in part to methods for producing biobased hydroxyl-terminated polyester polymers, wherein molten or hydrocarbonaceous fluid-dispersed polyhydroxyalkanoate (PHA) polymers are reacted with a difunctional hydroxylated insertion compound at a sufficient temperature, with or without a catalyst and for a sufficient time to depolymerize the starting PHA polymer to provide a hydroxyl-terminated reaction product of reduced molecular weight as compared to the starting PHA polymer. The difunctional hydroxylated insertion molecule (also referred to herein as an insertion compound) can be at least one of a diol, a polyhydroxy molecule having at least one primary hydroxyl group for insertion, and at least one other primary or secondary hydroxyl group, and aminoalcohol. When an aminoalcohol is used as the insertion compound, a catalyst is often not needed as the amide formation drives the reaction to completion, however, if a hydroxyl group is used for insertion, then a transesterification catalyst may be required to increase the rate of conversion. The catalyst can be Lewis acid or Lewis base-type with the preferred being polyvalent metal catalysts from the transition elements in the Periodic Table of the Elements can be organo tin, organo zirconium, and titanoium based, with the most common being at least one of dibutyl tin dilaurate (DBTL), dibutyl tin oxide (DBTO), or zinc acetate. The hydroxyl-terminated reaction products can be polyester diols or polyester polyols having hydroxyl functionality at one or both ends of the polymer chain. As an option, ester groups can be cleaved in the starting PHA polymer to produce a hydroxyalkanoate ester on one side of the polymer chain and a hydroxyl functionality attached to the alkanoic acid group on the other side of the polymer chain. As an option, the hydroxyl-terminated reaction product can be at least one of hydroxyl-terminated polyhydroxybutyrate and hydroxyl-terminated polylactic acid, depending on the starting polymer. As an option, the hydroxyl-terminated reaction product has a molecular weight from about 200,000 to about 250, and the molecular weight of the starting polymer that is depolymerized can be at least about four times greater, or at least about 10 time greater, or at least about 20 times greater than the reaction product polymers. The use of catalyst in the depolymerization reaction can be reduced or eliminated in some options. As an option, a liquid carrier may be used to facilitate easier mixing between the insertion molecule and the polymer, especially when the preferred reaction temperature is below the Tm (melting point) of the polymer. The liquid may be just an inert material, such as a hydrocarbon fluid, such as heptanes, octane, dodecane, or white mineral oil, which provides a stable uniform temperature for the mixture without being involved specifically with the reaction. Another variation is to use a solvent for the polymer which is non-reactive with the insertion molecule, e.g., a chlorinated hydrocarbon. Another alternative is to use two non-solvents for the polymer as a reaction mixture, for example, butanol is not a solvent for PHAs, neither are aminoalcohols; however, this blend of non-solvents at the reaction temperature can actually dissolve or rapidly disperse the PHA such that reaction conversion is accelerated and side reactions minimized and yield markedly improved conversion rates not possible with either component alone or as expected additively from them.

The present invention further relates to polyhydroxyalkanoate polyols having a molecular weight from about 200,000 to about 250 produced by the controlled/systematic depolymerization of high molecular weight biobased polyhydroxyalkanoate derived polymers with a difunctional hydroxylated insertion compound, wherein the molecular weight of starting polyhydroxyalkanoate is at least about 4 times greater than that of polyhydroxyalkanoate polyol reaction products of the depolymerization.

The present invention further relates to a method for making polyurethanes, wherein the hydroxyl-terminated reaction product is reacted with polyfunctional isocyanate to form a polyurethane product.

The present invention further relates to products, such as compositions and articles, containing or formed from biobased hydroxyl-terminated polyhydroxyalkanoate reaction products of the present invention. These products may include, for example, polyurethanes, macroblock polyesters, plasticizers, hydrophobe for nonionic surfactants, confectionary additives and foods, pharmaceuticals, and the like. The present invention further relates to products containing or formed from the polyurethanes of the present invention, such as foams, elastomers, adhesives, coatings, textiles, and the like.

It is to be understood that both the foregoing general description and the following detailed description are exemplary and explanatory only and are intended to provide a further explanation of the present invention, as claimed.

### DETAILED DESCRIPTION OF THE PRESENT INVENTION

The present invention relates in part to providing biobased polyester diols and polyols that are derived in part by a controlled/systematic depolymerization of biobased polyesters such as polyhydroxyalkanoate polymers or copolymers that have markedly higher starting molecular weights than the polyester diol and polyol reaction products. Instead of trying to build-up structure through chemical reactions to arrive at a desired PHA polymer product at a desired molecular weight, the methods of the present invention provide a highly-controllable system for breaking down long chain starting PHA polymers into end-capped PHA chain fragments of the desired structures and at the desired molecular weights as reaction products. These polymeric reaction products of the depolymerization reactions of the methods of the present invention can retain a high degree of intactness of the original chain structure other than at the end groups, which are capped. Difunctional hydroxylated insertion compounds, such as a diol, aminoalcohol, or polyhydroxyalcohol, can be used to insert into the original polymer chain of the starting PHA polymer. The insertion compounds also are able to react with the end moieties of the liberated chain fragments to cap the ends of the chains and concurrently introduce functional groups. The functional groups introduced at the chain ends can provide reactive sites for further reactions useful in the manufacture of PHA derivatives, such as polyurethanes or other materials. The polyhydroxyalkanoate products of the depolymerization reaction of the methods of the present invention thus can be highly useful intermediates. Depending on the insertion compound, hydroxyl-terminated PHA's can be produced by the depolymerization reactions in methods of the present invention which have a primary hydroxyl group on only one chain end or on both chain ends. As an option, the insertion compounds alternatively can be used to selectively cleave the starting PHA polymers to produce a hydroxyalkanoate ester on one side of the polymer chain and a hydroxyl functional group attached to the alkanoate acid group on the other side of the polymer chain. These variable outcome options can be controlled by selection combinations of starting polymer, insertion compound, catalyst, reaction mediums, and/or reaction conditions such as described and illustrated herein. Compositions and articles can directly contain or be formed at least in part with the biobased, high molecular weight polyester diols and polyols reaction products of the depolymerization reactions of the methods of the present invention.

As an option, biobased polyester diols and polyols can be derived in part from depolymerization reactions conducted on even higher molecular weight biobased polyhydroxyalkanoate polymers or copolymers (e.g., the starting polyester polyols) with the difunctional hydroxylated insertion compound and heating at a sufficient temperature and for a sufficient time to depolymerize the starting polymer. The reaction products then can be recovered from the reaction mixture. As an option, the depolymerization reactions can involve hydrolytic chain scission reactions and the use of a catalyst.

As an option, biobased polyester diols and polyols which, for example, are PHA diols and polyols, can be derived in part from biobased higher molecular weight PHA polymers and copolymers by a bulk melt-phase transesterification method or a hydrocarbonaceous fluid (nonaqueous) dispersion based transesterification method in different depolymerization options of methods of the present invention. Production of biobased hydroxyl-terminated polyester polymers with the synthesis routes offered in various methods of the present invention can provide numerous benefits and advantages. In bulk melt-phase processing, for example, the starting polyester polymer initially can be melted and then can be reacted with the insertion compound. Melting of starting polyester polymer can involve heating a solid form of the polymer (e.g., pellets, films, and so forth) to a temperature (such as at or above the melting temperature of the polymer) and for a time sufficient to provide a flowable molten form of the polyester polymer. The polyester polyol can be made sufficiently flowable in a molten state such that the difunctional hydroxylated insertion compound is substantially uniformly distributable therein, such as with mixing. The depolymerization reaction temperature used on the melt-phase reaction system can be varied to some extent depending on the PHA and insertion compound reactants used and reaction time period to be used. The depolymerization reaction can be carried out for some PHA's, for example, with heating of the polymer and insertion compound in contact with each other at a temperature of from about 150° C. to about 190° C. for from about 2 minutes to about 120 minutes. Higher reaction temperatures and longer reaction times typically correlate with greater depolymerization and fragmentation of the starting polymer into progressively smaller chains. As an option, the melt-phase processing can be carried out using a twin screw extruder, brabender mixer, or short mixing extruder directly into a heated stirred tank with inert gas blanket. Also, optionally, a catalyst can be added to the PHA polymer to speed up the rate of the transesterification reaction. The catalyst can be added into the molten PHA by itself. It can also be premixed with the insertion compound, then the mixture added to the molten PHA or finally the catalyst can be premixed with the PHA polymer along with a small amount (0.1%, 0.5% or 1% by weight) of the insertion compound prior to mixing and the balance of insertion compound added when the PHA polymer and catalyst are in the molten stage during mixing.

In hydrocarbonaceous fluid (nonaqueous) dispersion based transesterification methods used for the depolymerization reactions, the polyester polymer is dispersed in a liquid hydrocarbonaceous medium. The liquid hydrocarbonaceous medium can be nonaqueous or essentially nonaqueous (e.g., 0-2 wt % total water content). As an option, the liquid hydrocarbonaceous medium comprises liquid hydrocarbonaceous solvent which dissolves the polyester polymer and is substantially inert with respect to the depolymerization reactions. As an option, the hydrocarbonaceous solvent can be chloroform, dichloromethane, dioxane, tetrahydrofuran, dimethyl formamide, dimethyl sulfoxide, or any combinations thereof. As another option, the liquid hydrocarbonaceous medium comprises a heat transfer fluid, such as, for example, toluene, white mineral oil, or any combinations thereof. As with melt-phase processing, the reaction mixture in the hydrocarbonaceous fluid dispersion-phase processing is heated to a temperature and for a time sufficient, optionally with a catalyst present, to provide the depolymerization reactions, and then PHA reaction products can be recovered therefrom.

The methods of the present invention, in some options such as those which involve bulk melt-phase transesterification reactions, can avoid inherent production capacity limitations of depolymerization reactions otherwise conducted on high molecular weight polyester polyols as dissolved in very large excesses of short chain diols, such as propylene glycols. Other methods of the present invention in some options, such as those which involve hydrocarbonaceous fluid dispersion-phase based transesterification reactions, can provide greater control over reaction temperature and/or expand the useful range of reaction conditions by use of hydrocarbonaceous media that can be nonaqueous solvents or heat transfer fluids. The option of using butanol and aminoalcohol blends as the hydrocarbonaceous medium in a nonaqueous fluid-dispersion based processing option for the depolymerization reaction is significant since neither butanol itself nor aminoalcohols typically dissolve PHA's, but it has been found their blends can in the depolymerization reactions of methods the present invention. For these nonsolvent blends, the butanol and aminoalcohol can be blended in a weight ratio (w:w) of from about 1:10 to about 10:1, or other ratios. The butanol and aminoalcohol blend can be added to the reaction mixture in an amount of from about 50% to about 400%, based on weight of the starting polyester polymer. As options, the blend can be preblended before combination with the polyester polyol, or added separately to the reaction mixture containing the polyester polymer with mixing. It is not believed that this result can be reliably forecasted as the solubility of individual solvents or blends in the depolymerization reaction systems that are involved typically cannot be used to reliably predict solubility of reaction components in a candidate solvent therein, such as semicrystalline solvent. Solvents which may be expected to work, such as ethyl acetate, do not necessarily function as may be predicted. The methods of the present invention can provide one or more of increased bulk production capacity, accelerated conversion rates (shorter reaction times), increased process control and process ease, customization of the product structures and purities thereof, or other improvements and advantages.

In some options of the present invention, such as where a primary hydroxyl, or primary or secondary amine group, on the difunctional hydroxylated insertion compound is used for the insertion reaction, no transesterification catalyst is needed or its amount can be reduced in the depolymerization reaction. The avoidance of catalyst can reduce process steps needed, as it typically must be separated and recovered from the PHA reaction products before they can be further used. In many instances, catalyst needs to be removed as it can influence other reactions, e.g., urethane formation and can also promote further depolymerization or polymerization depending on the temperature conditions.

As an option, a high molecular weight polyhydroxyalkanoate (“PHA”) which can be used as the starting polyester polymer for depolymerization reactions of the methods of the present invention can have a repeat unit of the following general structure (I):

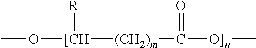

wherein m=0, 1, 2, 3, 4, 5, 6, 7, or 8; n can range from about 100 to about 150,000, or from about 250 to about 100,000, or from about 500 to about 50,000, or from about 1,000 to about 15,000, or from about 1,250 to about 10,000, or other values; and R is H or straight-chain or branched alkyl or alkenyl (e.g., C1-C10 alkyl or alkenyl). As an option, the value of n, together with the values of m and R, can be sufficient to provide an overall PHA polymer molecular weight of at least about 225,000 or more. Examples of repeat units include, for example, 3-hydroxybutyrate, 3-hydroxypropionate, 3-hydroxyvalerate, 3-hydroxyhexanoate, 3-hydroxyheptanoate, 3-hydroxyoctanoate, 3-hydroxynonaoate, 3-hydroxydecanoate, 3-hydroxydodecanoate, 3-hydroxytetradecanoate, 3-hydroxyhexadecanoate, 3-hydroxyoctadecanoate, 4-hydroxybutyrate, 4-hydroxyvalerate, 5-hydroxyvalerate, 6-hydroxyhexanoate, and lactic acid (lactide). As an option, a single type of starting PHA polymer can be used or alternatively, a physical blend of two, or three, or four, or five or more different types of starting PHA polymers can be used as the starting polyester polymer for depolymerization reactions of the methods of the present invention.

As an option, a PHA can be a homopolymer (all, or essentially all (e.g., greater than 98 wt %, or greater than 99 wt %, or greater than 99.5 wt %), monomeric repeat units are the same). Examples of PHA homopolymers include, for example, poly(3-hydroxybutyrate)(“P3HB”) or other poly-3-hydroxyalkanoates (e.g., poly-3-hydroxypropionate, poly-3-hydroxyhexanoate, poly-3-hydroxyheptanoate, poly-3-hydroxyoctanoate, poly-3-hydroxydecanoate, poly-3-hydroxydodecanoate), poly(4-hydroxybutyrate) (“P4HB”) or other poly-4-hydroxyalkanoates, poly-5-hydroxyalkanoates (e.g., poly-5-hydroxypentanoate); poly-6-hydroxyalkanoates (e.g., poly-6-hydroxyhexanoate), polylactic acid (“PLA”), polyhydroxyvalerate (“PHV”), polyglycolic acid (“PGA”), polycaprolactone (“PCL”), polyhydroxypropanoate (“PHPp”), polyhydroxypentanoate (“PHPt”), polyhydroxyhexanoate (“PHHx”), polyhydroxyheptanoate, (“PHHp”), and polyhydroxyoctanoate (“PHO”).

As an option, a PHA can be a copolymer (contain two or more different monomer units). Examples of PHA copolymers include, for example, poly(3-hydroxybutyrate-co-4-hydroxybutyrate) (“P3HB-co-P4HB”), poly(3-hydroxybutyrate-co-4-hydroxyvalerate) (“P3HB-co-PHV”), poly(3-hydroxybutyrate-co-3-hydroxypropionate), poly(3-hydroxybutyrate-co-3-hydroxyvalerate), poly(3-hydroxybutyrate-co-3-hydroxyhexanoate), poly(3-hydroxybutyrate-co-6-hydroxyhexanoate), poly(3-hydroxybutyrate-co-3-hydroxyheptanoate), poly(3-hydroxybutyrate-co-3-hydroxyoctanoate), poly(3-hydroxybutyrate-co-3-hydroxydecanoate), poly-(3-hydroxybutyrate-co-3-hydroxydodecanotate), poly(3-hydroxybutyrate-co-3-hydroxyoctanoate-co-3-hydroxydecanoate), poly(3-hydroxydecanoate-co-3-hydroxyoctanoate), and poly(3-hydroxybutyrate-co-3-hydroxyoctadecanoate). The PHA copolymers can be, for example, alternating, periodic, random, statistical, and block co-polymers thereof. Although examples of PHA copolymers having two different monomer units have been provided, a PHA can have more than two different monomer units (e.g., three different monomer units, four different monomer units, five different monomer units, six different monomer units, seven different monomer units, eight different monomer units, nine different monomer units, etc.).

As an option, the PHA polymer used as the starting material for the depolymerization reaction can be a homopolymer or copolymer of polyhydroxybutyrate or a polylactic acid, used singly or in any combinations thereof. The polyhydroxybutyrate can be, for example, a homopolymer such as poly(3-hydroxybutyrate) (P3HB), poly(4-hydroxybutyrate) (P4HB), used singly or in any combinations thereof. The PHA can be a copolymer, such as a copolymer of 3-hydroxybutyrate and 4-hydroxybutyrate as poly(3-hydroxybutyrate-co-4-hydroxybutyrate) (P3HB-co-P4HB). The polyhydroxybutyrate copolymers can be, for example, alternating, periodic, random, statistical, and block co-polymers thereof. The polylactic acid can be any isomers thereof, such as D-polylactic acid (poly(D-lactide)), L-polylactic acid (poly(L-lactide)), used singly or any combinations thereof.

As an option, the starting polyester polymer used in depolymerization reactions of methods of the present application can have a weight average molecular weight (M_{w}) of at least about 225,000, or at least about 400,000, or at least about 500,000, or at least 600,000, at least about 800,000, or at least about 1,000,000, or at least 2,000,000, or at least about 3,000,000, or at least about 5,000,000, or at least about 10,000,000, or at least about 15,000,000 or higher values, or from about 225,000 to about 20,000,000, or from about 400,000 to about 15,000,000, or from about 500,00 to about 10,000,000, or from about 600,000 to about 5,000,000, or from about 700,000 to about 3,000,000, or from about 800,000 to about 2,000,000, or other values. As an option, the starting polyester polymer, e.g., the indicated high molecular weight PHA's, can be derived from microbial fermentation of sugars or lipids. Conventional bacterial fermentations reactions used to produce PHA's can be applied in this respect. As an option, the PHA's can be derived from biomass sources, such as described in U.S. Patent Application Publication Nos. 2003/0158274 A1, and above-cited literature references of Lee, Braunegg et al., and Madison et al., which all are incorporated herein by reference in their entireties. As used herein, “molecular weight” refers to weight average molecular weight (M_{w}). Weight average molecular weight can be determined by gel permeation chromatography, such as using a Waters Alliance HPLC System equipped with a refractive index detector and autosampler. The column set is a series of three PLGel 10 μm Mixed-B (Polymer Labs, Amherst, Mass.) columns with chloroform as mobile phase pumped at 1 ml/min. The column set is calibrated with narrow distribution polystyrene standards.

As an option, the methods of the present invention can provide biobased polyester diols and polyols that are derived in part by a controlled/systematic depolymerization of higher molecular weight biobased PHA's, wherein a repeat unit of the original PHA can be essentially maintained throughout the resulting polymer chain fragments other than at end-chain groups of the polyester diol and polyol products.

Thus, as an option, the repeat units of the biobased polyester diols and polyols produced with depolymerization reactions of the present invention also can be represented by the indicated general structure (I) with the number of repeat units n reduced in value as compared to the parent (starting) PHA polymer, and reference is made thereto. The changed n value for the PHA reaction products of the depolymerization reaction can be represented as “n*” herein. For example, as an option, where n represents the number of repeat units in the parent (starting) PHA polymer with reference to general structure (I), and n* represents the number of the same repeat units in the hydroxyl-terminated PHA polymer reaction product of the depolymerization reaction, the numerical ratio of n/n* can be least about 4, or at least about 10, or at least about 20, or higher values (e.g., where n is 10,000 and n* is 1,000, then the ratio n/n* would 10/1 or a factor of 10).

As an option, the polyhydroxyalkanoate reaction product of the depolymerization reaction of methods of the present invention can have the following structure (II):

H—(O—CHR¹—(CH₂)ₘ—C(O)—)_{n*}OR²

wherein for the hydroxyalkanoate polymer, for instance, n* can be values as indicated, R¹ is a straight chain or branched chain alkyl (e.g., C1-C10 alkyl or alkenyl), m is 0-8, and R² is H, and wherein for hydroxyalkanoate ester polymer, for instance, n* can be values as indicated, R¹ and m can have the same indicated values, and R² can be methyl, ethyl, propyl, isopropyl, butyl, isobutyl, pentyl, hexyl, 2-ethyl hexyl, or other alkyl groups having at least one primary or secondary hydroxyl group present on the end. The “—C(O)—” in the structure represents a carbonyl.

As another option, the polyhydroxyalkanoate reaction product of the depolymerization reaction of methods of the present invention can have the following structure (III):

H—(O—CHR¹—(CH₂)ₘ—C(O)—)_{n*}NR²

wherein for the hydroxyalkanoate polymer, for instance, n* can be values as indicated, R¹ is a straight chain or branched chain alkyl (e.g., C1-C10 alkyl or alkenyl), m is 0-8, and R² is H, and wherein for hydroxyalkanoate ester polymer, for instance, n* can be values as indicated, R¹ and m can have the same indicated values, and R² can be methyl, ethyl, diethyl, propyl, isopropyl, butyl, isobutyl, pentyl, hexyl, 2-ethyl hexyl, or other alkyl groups having at least one primary or secondary hydroxyl group present on the end. The “—C(O)—” in the structure represents a carbonyl.

As an option, the hydroxyl-terminated PHA reaction products that can be produced with depolymerization reactions of the present invention can have a weight average molecular weight (M_{w}), for example, of from about 200,000 to about 250, or from about 150,000 to about 500, or from about 100,000 to about 750, or from about 75,000 to about 1,000, or from about 60,000 to about 2,000, or from about 50,000 to about 3,000, or from about 40,000 to about 4,000, or from about 30,000 to about 5,000, or from about 25,000 to about 7,500, or from about 20,000 to about 10,000, or from about 17,500 to about 12,000, or other values provided that the molecular weight is smaller than that of the starting polymer that is depolymerized. The hydroxyl-terminated reaction products of the present invention can have markedly smaller molecular weights than the starting polymer while, as an option, can be prepared having relatively high molecular weights by controlling the reactant mixture composition and reaction conditions of the depolymerization reaction of methods of the present invention. As an option, the starting polymer can have a molecular weight that is at least about 4 times, or least about 5 times, or at least about 10 times, or at least about 15 times, or at least about 20 times, or from about 4 to about 20 times, or from about 5 to about 15 times, greater than the molecular weight of the hydroxyl-terminated reaction products of the depolymerization reactions of the present invention.

The difunctional hydroxylated insertion compound can be, for example, an aliphatic diol, an aminoalcohol, or a polyhydroxyalcohol. As used herein, the term “hydroxylated” means the compound includes at least one hydroxyl (—OH) functional group. As an option, the insertion compound includes at least one primary hydroxyl group, and may have two primary hydroxyl groups. As an option, the insertion compound has a molecular weight less than about 250 and has at least one primary hydroxyl group or amine group designed to insert into the polymer chain and at least one additional hydroxyl group which has the same or different functionality to the hydroxyl group on the alkanoate repeat unit. The short or medium chain aliphatic diol can be, for example, a C3 chain, C4 chain, or C5 chain aliphatic diol, which includes at least one primary (i.e., end chain) hydroxyl group. In various options, the short or medium chain aliphatic diol can have two primary hydroxyl groups, or one primary hydroxyl group and one secondary hydroxyl group. The aminoalcohol can be, for example, a dialkanolamine such as a short or medium chained (e.g., C2 to C5 hydroxylated chain segments) dialkanolamine. The dialkanolamine can be, for example, diethanolamine (HN(CH₂CH₂OH)₂)), which is polyfunctional as a secondary amine and a diol. The aminoalcohol also can be, for example, a short or medium (e.g., C4-C6) branched chain aminoalkanol having a single (primary) hydroxyl group. The aminoalcohol can be for example, 2-amino-2-methyl propanol ((CH₃)₂C(NH₂)CH₂OH). As an option, the aminoalcohols can have the effect of irreversibly or essentially irreversibly reacting with ester groups to systematically reduce the molecular weight of the parent (starting) PHA polymer. The difunctional hydroxylated insertion compound can be, for example, at least one of 1,2-propanediol (propylene glycol), dipropylene glycol, 1,3-propanediol, 1,2-butanediol, 1,3-butanediol, 1,4-butanediol, trimethylolpropane, diethanolamine, 2-amino-2-methyl propanol, glycerol, sorbitol, or any combinations thereof. As an option, the insertion compound is an aminoalcohol having either a primary or secondary amine functionality and at least one primary or secondary hydroxyl. Examples being ethanolamine, diethanolamine amino 1-propanol, amino 2-propanol, 2 amino-2-methyl propanol, and the like. As an option, the insertion compound is a polyhydroxyalcohol having at least one primary alcohol, such as glycerol, propylene glycol, dipropylene glycol, sorbitol, and the like. Optionally, it may be desirable to use a single type of difunctional hydroxylated insertion compound in the depolymerization reactions of the methods of the present invention for reducing process variables and providing greater control with respect to the structures of the reaction products. In other options, the use of combinations of different difunctional hydroxylated insertion compounds may be useful in the depolymerization reactions.

In options of the depolymerization reactions of the present invention, the difunctional hydroxylated insertion compound can be used, for example, in a stoichiometric equivalent amount or a greater amount (i.e., stoichiometric excess) in the depolymerization reactions. This stoichiometry of the difunctional hydroxylated insertion compound is referenced to the amount thereof required to produce the desired (targeted) number of hydrolytic chain scissions to react to the desired or targeted molecular weight value or molecular weight range or distribution. Further, if the hydroxyl insertion group is the same as the ester linkage, then cleaving is needed and then statistics will determine if it will happen with no thermodynamic driver. Excess insertion group can be used as this will drive the reaction to completion more rapidly, leaving the residual diol or aminoalcohol is optional but not preferred as this can influence the final properties of the system. As an option, the total amount of difunctional hydroxylated insertion compound added can be in the range of from about 1 to about 5 times, or from about 1 to about 4 times, or from about 1 to about 3 times, or from about 1 to about 2 times, or from about 1.1 to about 5 times, or from about 1.25 to about 4 times, or from about 1.4 to about 3 times, or from about 1.5 to about 2.5 times, or from about 1.5 to about 2 times, the stoichiometric amount.

Based on the type of starting polyester polymer and difunctional hydroxylated insertion compound selected for the deploymerizing reaction, the concentration of the insertion compound and control of the reaction conditions (e.g., reaction temperature, time), the reaction products obtained can be tailored or customized with an increased level of predictability and purity in options of the present invention. As an option, when the polyester polymer comprises poly(3-hydroxybutyrate-co-4-hydroxybutyrate), the use of a difunctional hydroxylated insertion compound which comprises at least one primary hydroxyl group and at least one secondary hydroxyl group (e.g., 1,2-propanediol, 1,2-butanediol, 1,3,-butanediol, diethanolamine, 2-amino-2-methyl propanol) is capable of forming hydroxyl terminal groups on the hydroxyl-terminated poly(3-hydroxybutyrate-co-4-hydroxybutyrate) reaction product, which can have approximately equal reactivity towards isocyanate groups when incorporated into polyurethanes. As an option, when the polyester polymer comprises poly-4-hydroxybutyrate, the use of a hydroxylated insertion compound which comprises primary hydroxyl groups only (e.g., 1,3-propane diol, 1,4-butanediol, trimethylolpropane, 1,6-hexanediol, and the like) is capable of forming primary hydroxyl terminal groups on the hydroxyl-terminated poly-4-hydroxybutyrate reaction product, which can have approximately equal reactivity towards isocyanate groups when incorporated into polyurethanes.

As an option, poly-3-hydroxybutyrate may be reacted with ethanolamine as the insertion molecule to provide a poly-3-hydroxybutyrate chain with a secondary hydroxyl group at one end and an ethanolamide linkage through the terminal carboxylic acid, providing a polymer with primary and secondary functionality. Similarly, if diethanolamine is used in preference to ethanolamine then a diethanolamide can be the resulting terminal group, and in this case the PHA polyols have two primary hydroxyl groups and one secondary hydroxyl groups thus making it useful in branched polyurethane compositions. Further, if propylene glycol is used as a replacement for ethanolamine, then the primary hydroxyl group on the 1,2-propanediol inserts into the butyrate ester and the resulting polyol now has statistically two secondary hydroxyl groups whilst if 1,3-propanediol or butanol are used, the PHA polyols have a primary and secondary terminal hydroxyl groups. Furthermore, if glycerol is used as the insertion molecule, then a trihydroxyl PHA polyol with one primary hydroxyl and two secondary hydroxyl groups is produced.

In greater detail, the bulk-melt phase processing and hydrocarbonaceous fluid-dispersion phase processing of depolymerization reactions of the methods of the present invention can be further illustrated in a non-limiting manner with reference to the processing of polylactic acid (“PLA”), and polyhydroxybutyrates, such as poly(3-hydroxybutyrate-co-4-hydroxybutyrate) (P3HB-co-P4HB) copolymer and poly-4-hydroxybutyrate (P4HB). As an option, routes to make P3HB-co-4HB polyols and P4HB polyols from high molecular weight polymers can involve inserting a more reactive group into the polymer chain to cleave the current ester group by using a primary hydroxyl group or primary or secondary amine group. In both cases, the insertion molecule also has a second hydroxyl functionality such that the resulting reduced molecular weight polymer has hydroxyl functionality at both ends of the polymer chain.

In production of P3HB-co-4HB polyols with high 3HB content (e.g., 95 to 50 wt % 3HB content), a bulk transesterification process can be involved whereby the polymer is converted into a molten form at around 150-180° C. and the insertion compound, such as propylene glycol, with predispersed/predissolved catalyst, if used, can be added directly into the molten polymer with good mixing to form a homogeneous fluid. The fluid mix can then be maintained at the reaction temperature for the desired time to convert to a lower molecular weight P3HB-co-4HB diol with terminal secondary hydroxyl groups. The amount of propylene glycol or other insertion compound added can be in the range, for example, of 1-2 times the stoichiometric amount required to produce the desired number of hydrolytic chain scissions to react the target molecular weight. Depolymerization catalysts, if used, can be, for example, DBTL, DBTO and zinc acetate. DBTL can also act as a catalyst for isocyanate-hydroxyl reactions (e.g., where these P3HB-co-4HB diol products are used in polyurethane production), whilst neither DBTO nor zinc acetate influence isocyanate-hydroxyl reactions. Thus, residual catalyst content or contamination in the PHA depolymerization reaction products can be undesirable. Catalyst content can be adjusted, for example, from about 0.1 wt % to 10 wt % based on propylene glycol content. Total reaction time can be, for example, from about 5 minutes to around 90 minutes depending on the molecular weight of the diol required. For example, using 20% propylene glycol with 5% DBTL in P3HB-co-11%-4HB with starting molecular weight of ab out 650,000, a diol with molecular weight of about 30,000 (solid where cooled to room temperature) can be achieved in about 5 minutes reaction time and about 5,000 (liquid at room temperature) after about 70 minutes at about 170° C. Reaction insertion is primarily through the primary hydroxyl group on propylene glycol producing a diol with two secondary hydroxyl groups. The reaction can be carried out using a twin screw extruder, brabender mixer as the primary reaction vessel or a short mixing extruder directly into a heated stirred tank with nitrogen blanket. Diol can have very low viscosity at the reaction temperature (1 to 500 Pas), depending on molecular weight, such that it can easily be pumped into a final storage container or through a discolorizing filtration unit. Diols produced by this route can be readily converted into biobased urethane products through reaction with a difunctional or polyfunctional isocyanate component (e.g., TDI, MDI, pMDI, IPDI, and the like). An advantage of the indicated process for producing the P3HB-co-4HB copolymer lies in a faster reaction conversion which can easily be monitored through inline viscosity measurement tools (low melt viscosity will not plug units) compared with the previous process, and minimal post reaction work up. The process is ideally suited for polyol insertion that has at least one primary and at least one secondary hydroxyl e.g., 1,2-propanediol, 1,2-butanediol, 1,3-butanediol, and the like.

As indicated, a P3HB-co-4HB diol can be used with at least one primary and one secondary hydroxyl group such that the P3HB-co-4HB diol can have equal functionality at both chain ends, and hence can have equal reactivity towards isocyanate groups, which can be relevant to polyurethane type reactions or others. As an option, therefore, the transesterification reaction P3HB-co-4HB can b e designed to insert through the primary hydroxyl group on the diol and not secondary hydroxyl. P4HB has a primary hydroxyl group as the terminal hydroxyl group for each repeat unit, such that the approach used in P3HB-co-4HB diol does not directly translate to P4HB for good chain breakdown and to have a final product with equal reactivity. The polyol choice can be changed to PH4B, for example, such that all the hydroxyl groups are preferably primary in nature, e.g., diols such as 1,3-propanediol, butanediol, trimethylol propane, hexanediol, and the like. Further, P4HB is inherently more thermally stable than high 3HB copolymers and so the processing temperatures can be increased without the risk of beta-elimination reactions generating potentially undesirable crotonate (vinyl) end groups. There is however the possibility of producing P4HB-diol and P4HB-diol-P4HB, both of which are suitable for many end use applications. The manufacturing process can involve premelting a starting P4HB polymer (e.g., having a molecular weight of about 426 k or other high MW values) at about 170-180° C., for example, and then adding the insertion compound, e.g., 1,3-propanediol at about 10%, for example, based on P4HB to form a homogeneous mixture in a batch mixer, such as a brabender. The viscosity of this mixture can remain very stable, such as for about 10-15 minutes at 180° C. A catalyst, e.g., DBTO, DBTL, zinc acetate, and the like, can be added, for example, at about 0.1% to 10 wt % based on the 1,3-propanediol content, which initiates the transesterification depolymerization with a reduction in viscosity and molecular weight. A sample removed after a few minutes (e.g., about 5 minutes), which after cooling can become a hard waxy solid whilst after about 15 minutes a permanently low viscosity liquid can be generated. This molecular weight reduction reaction can be extremely rapid when compared with a P3HB-co-4HB (e.g., 11% 4HB copolymer) polymer where the conversion to a liquid product can take, for example, about 70 minutes. This route can be repeated using other insertion compounds having primary hydroxyl groups, such as 1,4-butanediol, instead of 1,3-propanediol with similar results of a solid formed after about 5 minutes and a liquid after about 15 minutes. A continuous process can be carried out whereby the P4HB is premixed with the catalyst (1%), using about 0.5% diol to provide wetting of the catalyst onto the polymer pellets and avoiding segregation, and feeding the wetted polymer pellets into the rear throat of a twin screw extruder, such as at a rate of 2 lb./hr and a flat barrel temperature profile of 170° C.; and the remaining 1,3-propanediol can be pumped into a melt 2LID section from an initial feeding port such that the final ratio of P4HB to diol can be, for example, 90/10. The feed rate and screw rpm (25 pm) can control the residence time in the extruder to around 15 minutes and a low viscosity liquid viscosity similar to water) can be discharged, which can become solid, such as within 10 minutes of cooling. The extruder rpm can be reduced to about 10 rpm to produce a residence time of around 20-25 minutes, for example, and a permanent liquid diol can be formed.

Solvents, such as chloroform, dichloromethane, dioxane, tetrahydrofuran, dimethyl formamide, dimethyl sulfoxide in most cases are good solvents for P4HB (and P3HB-co-4HB copolymers) and the selection of the solvent boiling point can be used to control reaction temperature. At the end of the conversion, the diol typically needs to be separated from the solvent by precipitation, vacuum distillation, other solvent partitioning to remove catalyst impurities, or the like. Alternatively, a nonaqueous, nonsolvent liquid may be used as a heat transfer medium, such as for P4HB, where the melting point of the polymer is relatively low. In these cases, carrier liquids, such as toluene or white mineral oil, may be used. The same catalyst types used for melt transesterification can be used for solution or dispersion reactions, e.g., DBTL, DBTO, zinc acetate and the like. Hence, solvents or processes can be used which are more suitable for CGMP and pharmaceutical applications can be selected and the reaction rapidly quenched. Products can be made more suitable for oral consumption, e.g., using these solid but low melting point P4HB diols, such as by mixing them with an orally acceptable plasticizer, processing aid, and the like, such as used in chewing gum manufacturing processes, e.g., high frucose corn syrup.

As an option, PLA (polylactic acid) can be used to replace the P3HB-co-4HB, P4HB, or P3HB polymers. This can produce a different range of polyester polyols for urethane reactions. The added advantage of this approach is that the PLA can be more thermally stable than P3HB derived polymers, which can be prone to beta-elimination reactions under higher temperatures and base conditions, hence the conversion reaction with either diols or aminoalcohols can be accomplished at temperatures closer to the melting point of the PLA (i.e., 160° C.) and above, thus having a much faster conversion rate and shorter reaction times. The solubility of PLA is also higher than P3HB polymers in a variety of solvents and so the solution reaction can also be carried out under a wider range of conditions. A further extension of this option is that by changing the stoichiometric ratio of the insertion polyol or aminoalcohol, then polymer oligomers with the same or different hydroxyl functionalities can be obtained. If the polyester repeat unit is, for example, from about 1 to 15 units, this can be used as a hydrophobe for nonionic surfactants, but with higher polarity that the traditional fatty acid or fatty alcohol starting material; thus providing additional surfactant products which have more rapid biodegradability than fatty acid/alcohol versions. Further, the PHA polyols which include lactic acid and the above-indicated blends of nonsolvents of butanol and aminoalcohol, when used in combination, increases the solubility parameter of the mix (polarity) to improve dissolution, which accelerates the reaction conversion compared with a heterophase erosion reaction. In examples conducted on polyols derived from P3HB-co-4HB (e.g., P3HB-co-11% 4HB) carried out using butanol as a suspending solvent in small heating blocks at about 93° C., for example, the polymer granules are only slightly swollen in the butanol over a multiple (e.g., about 7) hour time period. However, when an aminoalcohol (e.g., diethanolamine and 2-amino-2-methyl propanol both used separately) are added at molar ratios of aminoalcohol to the 3HB repeat unit and molecular weight of approximately >500,000 from about 0.05 to about 1:1, for example, the polymer can be rapidly dispersed in the butanol/aminoalcohol mix within about 15 minutes and clearly can aid the reaction conversion. Diols ranging from softer solids, through waxes and greases to liquids can readily be produced within a few hours time frame (e.g., about 5 or less hours). The PLA process can be carried out at higher temperatures than those allowed for P3HB polymers or copolymers because the beta elimination mechanism does not occur. Reaction temperatures do not need to be reduced to further minimize potential crotonate side reactions. PLA diol products also can easily recovered by precipitation in heptane and heptane/butanol mix could be recovered by fractional distillation.

The present invention also relates to polyurethanes which can be derived from the biobased, polyester diol and polyol products of the indicated depolymerization reactions of high molecular weight PHA's. Methods of producing these polyurethanes are also described. More specifically, a polyurethane can be or include the reaction product of: (a) at least one biobased, high molecular weight polyester diol or polyol reaction product of a depolymerization reaction such as described herein; (b) at least one isocyanate; (c) optionally a catalyst. In addition to catalysts, other optional ingredients for the polyurethane production can include, for example, crosslinkers, surfactants, blowing agents, and the like.

Regarding the at least one biobased, high molecular weight polyester diol or polyol, similar compounds to those indicated elsewhere herein for these compounds can be used as part of the reactants to form the polyurethane. One or more of these polyester diols or polyols can be used in the polyurethane synthesis. The amount of polyester diol or polyol present can be provided as a weight ratio compared to the isocyanate present. Specifically, the weight ratio (w:w) of isocyanate to the polyester diol/polyol can be, for example, from about 0.5:1 to about 2:1, respectively, or other amounts.

With regard to the at least one isocyanate, for purposes of the present invention, the at least one isocyanate can be or include an isocyanate-containing material. One or more isocyanates can be used in the reaction. The isocyanate containing material can be a polyisocyanate. The isocyanate or isocyanate-containing material can contain at least two isocyanate groups per molecule. The polyisocyanates can be any polyisocyanate traditionally used in the formation of polyurethanes. These polyisocyanates can be modified or unmodified versions. Preferably, the polyisocyanate is an aromatic polyisocyanate. A more specific example would be a toluene diisocyanate or mixtures containing toluene diisocyanate. The isocyanates can also be modified by other components, such as urethane, allophanate, uretdione, or other groups. The isocyanates described earlier can also be used. The isocyanate component can be or include a toluene diisocyanate, methylene 4,4′ diphenyl diisocyanate, isophorone diisocyanate, hexamethylene diisocyanate, or combinations thereof. Generally, the amount of isocyanate used would be the same as in the conventional making of polyurethanes. Examples of amounts are from about 5 to about 50% by weight of total reactants.

In more detail, the isocyanate can be a polyisocyanate, such as a molecule with two or more isocyanate functional groups, R—(N═C═O)_{n≧2}. The polyol can be a molecule with two or more hydroxyl functional groups, R′—(OH)_{n≧2}. The reaction product is a polymer containing the urethane linkage, —RNHCOOR′—. The isocyanate can be an aromatic, such as diphenylmethane diisocyanate (MDI) or toluene diisocyanate (TDI); or aliphatic, such as hexamethylene diisocyanate (HDI) or isophorone diisocyanate (IPDI). An example of a polymeric isocyanate is polymeric diphenylmethane diisocyanate, which is a blend of molecules with two-, three-, and four- or more isocyanate groups, with an average functionality of 2.7. Isocyanates can be further modified by partially reacting them with a polyol to form a prepolymer. A quasi-prepolymer is formed when the stoichiometric ratio of isocyanate to hydroxyl groups is greater than 2:1. A true prepolymer is formed when the stoichiometric ratio is equal to 2:1.

Other aromatic isocyanates include p-phenylene diisocyanate (PPDI), naphthalene diisocyanate (NDI), and o-tolidine diisocyanate (TODI). Examples of isocyanates include 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane (H₁₂MDI or hydrogenated MDI). Other aliphatic isocyanates include cyclohexane diisocyanate (CHDI), tetramethylxylene diisocyanate (TMXDI), and 1,3-bis(isocyanatomethyl)cyclohexane (H₆XDI).

One or more catalysts optionally can be present. Conventional catalysts used for the making of polyurethanes can be used in the present invention. Catalysts such as amine compounds or organometallic complexes can be used. The catalyst can be, for example, tertiary amines, such as dimethylcyclohexylamine or triethylamine. The organometallic compounds can be, for example, dibutyltin dilaurate or bismuth octanoate. Furthermore, catalysts can be chosen based on whether they favor the urethane (gel) reaction, such as 1,4-diazabicyclo[2.2.2]octane (also called DABCO or TEDA), or the urea (blow) reaction, such as bis-(2-dimethylaminoethyl)ether, or specifically drive the isocyanate trimerization reaction, such as potassium octoate. Other examples include organometallic compounds based on mercury, lead, tin (dibutyltin dilaurate), bismuth (bismuth octanoate), and/or zinc. Specific examples include mercury carboxylates, such as phenylmercuric neodeconate, alkyl tin carboxylates, oxides and mercaptides oxides, for example, dibutyltin dilaurate, dioctyltin mercaptide, or dibutyltin oxide, tin mercaptides.

One or more crosslinking agents optionally can be present. The crosslinking agent can be any crosslinking agent which is useful for polyurethane synthesis chemistries. The crosslinking agent can be, for example, polyols, which typically are polyethers. The polyols which can be used for crosslinking agents can be, for example, propylene glycol and trimethylolpropane combined with sucrose or polyesters, or ethylene glycol, 1,2-propanediol, 1,4-butenediol, and diethylene glycol combined with glycerol. Conventional crosslinking agents for polyurethane forming reactions may be used. The crosslinking agent also may be the reaction product of (i) one or more hydroxyalkanoates (e.g., 3-hydroxyalkanoate), such as 3-hydroxyalkanoate ester or 3-hydroxyalkanoate acid (e.g., 3-hydroxyalkanoate acid, 3-hydroxyalkanoate acid oligomer, or 3-hydroxyalkanoate acid polymer) with (ii) at least one amine having a single primary or secondary amine functionality and at least two hydroxyl groups having primary or secondary hydroxyl functionality. As an option, the hydroxyalkanoate or polyhydroxyalkanoate which can be used in forming the crosslinking agent in this respect can have the following formula:

H—(O—CHR¹—CH₂—C(O)—)ₙOR²

wherein for the hydroxyalkanoate ester, for instance, n=1, R¹═OR, where R is H, methyl, ethyl, propyl, butyl, pentyl, hexyl, octyl, or decyl, and R²=methyl, ethyl, propyl, isopropyl, butyl, isobutyl, pentyl, hexyl, or 2 ethyl hexyl; wherein for the hydroxyalkanoate oligomer, for instance, n=2 to about 20, R¹═OR, where R² is H, methyl, ethyl, propyl, butyl, pentyl, hexyl, octyl, or decyl; wherein for the hydroxyalkanoate polymer, for instance, n=21 to about 1000 or more, R¹═OR, where R² is H, methyl, ethyl, propyl, butyl, pentyl, hexyl, octyl, or decyl. In each case above for the oligomer or polymer, for instance, R²=ester or OH, such as a free acid or alkali/alkaline earth cation, such as sodium, potassium, or calcium. The hydroxyalkanoate, and in particular, the polymer, also may have a crotonate end termination. An exemplary structure is:

(CHR¹═CH—C(O)—)(OCHR¹CH₂—C(O)—)ₙOR²

wherein R¹, R², and n are as stated earlier in the previous formulas. The 3-hydroxyalkanoate ester which can be used in forming the crosslinking agent can be, for example, a methyl, ethyl, propyl, isopropyl, pentyl, hexyl, or 2-ethylhexyl ester of 3-hydroxyalkanoate. Further, the 3-hydroxyalkanoate acid may be a racemic 3-hydroxyalkanoate acid or a racemic 3-hydroxyalkanoate acid oligomer having from 2 to 10,000 repeat units and a terminal carboxylic acid end group. The amine which can be used in forming the crosslinking agent can be a dialkanolamine, for example, diethanolamine, tris(hydroxymethyl)amino methane, 2-aminoethyl 1,3 propane diol, 2 amino 1-methyl 1,3 propane diol, diisopropanolamine, diisobutanolamine, di-beta-cyclohexanolamine, or any combination thereof. The amount of the reactants (in the reaction to form the crosslinking agent) with regard to the hydroxyalkanoate component and the amine, can be present in equal molar ratios or about equal molar ratios, such as a molar ratio of 0.8:1 to 1:0.8.

The crosslinking agent may comprise, for example, greater than about 70% by weight 3-hydroxybutyrate diethanolamide, and/or from about 1 to about 10 wt % diethanolamine, and/or from 0.1 to 5 wt % alcohol, and/or less than 5 wt % alkanoic acid, alkanoic diethanolamide, and/or free 3-hydroxyalkanoate acid. As another option, the crosslinking agent can have the following diethanolamide generic structure:

H(OCHR¹CH₂—C(O)—)ₙ—N(CH₂CH₂OH)₂

where m=1 to 10, such as 1, 2, 3, 4, 5, 6, 7, 8, 9, or 10, and R¹ being as indicated previously. Other crosslinking agents that can be used herein can have similar structures. The crosslinking agent can be present in an amount, for example, of from one part per hundred (w:w) of the polyester polymer to about 10 parts per hundred (w:w) of the polyester polymer used in the reaction to form the polyurethane. For purposes of the present invention, it is understood that the crosslinking agent used in the present invention can alternatively or in addition be considered a chain extender.

One or more surfactants optionally can be present. Surfactants can be used to modify the characteristics of the polymer during the foaming process. They can be used to emulsify the liquid components, regulate cell size, and stabilize the cell structure to prevent collapse and surface defects. The surfactants can take the form of polydimethylsiloxane-polyoxyalkylene block copolymers, silicone oils, nonylphenol ethoxylates, and/or other organic compounds.

One or more blowing agents optionally can be used in the formation of the polyurethanes, if desired or needed for the end-use. Blowing agents activated chemically or by mechanical means optionally can be used in the present invention. Conventional blowing agents can be used, such as water and/or low-boiling inert liquids, such as hydrocarbons. The blowing agent also can be, for example, a pentane, such as a cyclopentane or n-pentane, certain halocarbons, such as HFC-245fa (1,1,1,3,3-pentafluoropropane) and HFC-134a (1,1,1,2-tetrafluoroethane), or can be combinations of various blowing agents. The blowing agent can be incorporated into the poly side or added as an auxiliary stream.

The catalysts, crosslinkers, surfactants, blowing agents, if used, can be present in conventional amounts. Other additives including conventional additives used for polyurethane manufacture also may be used. Other additives customary to polyurethane formulations can be used in the present invention including, but not limited to, flame retardants, foam stabilizers, fillers, antioxidants, pigments, and the like. These various additives can be used in conventional amounts, if present.

Reaction conditions and various components and amounts that can be adapted for use with the present biobased polyester polyols in polyurethane manufacture of the present invention are described, for example, in a variety of U.S. patents, including, but not limited to, U.S. Pat. Nos. 6,087,466; 6,087,410; 6,043,292; 6,034,149; and 6,087,409, all of which are incorporated in their entirety by reference herein. In general, the method of forming the polyurethane involves taking each of the components and mixing them together at room temperature. In making the polyurethanes of the present invention, the reactants can simply be mixed together under ambient conditions with low shear or high shear mixing. The reaction can occur in minutes or in hours depending on temperature and the optional use of catalyst.

The polyurethane can have a number of different properties. The polyurethane can be biodegradable and can be recycled. Further, the polyurethane can be used in a number of applications, including, but not limited to, coatings, foams (including rigid and flexible), elastomers, dispersions, and other water dispersible applications. The polyurethane can be formed into a number of articles, such as pipes, insulation, and any other articles traditionally formed from polyurethane materials such as dash boards, other automobile components, and the like. These various applications can be accomplished using conventional techniques known to those skilled in the art in view of the present application.

The polyester diol and polyol products of the present invention can be used in a wide variety of products, such as compositions and articles, containing or formed from the hydroxyl-terminated polyhydroxyalkanoates or polylactic acids of the present invention. These products can include, for example, not only the indicated polyurethanes, but also macroblock polyesters, plasticizers, hydrophobe for nonionic surfactants, confectionary additives and foods, pharmaceuticals, and the like.

Accordingly, the present invention includes the following aspects/embodiments/features in any order and/or in any combination:

1. The present invention relates to method of producing a biobased hydroxyl-terminated polyester, comprising: melting a high molecular weight polyester polymer comprising polyhydroxyalkanoate polymer, to provide molten polyester polymer; reacting the molten polyester polymer with difunctional hydroxylated insertion compound at sufficient temperature and for sufficient time to depolymerize the molten polyester polymer to provide at least one of hydroxyl-terminated reaction product having reduced molecular weight as compared to that of the molten polyester polymer, wherein the difunctional hydroxylated insertion compound is diol, aminoalcohol, or polyhydroxyalcohol, or any combination thereof, and the hydroxyl-terminated reaction product comprises hydroxyl-terminated polyhydroxyalkanoate.
2. The method of any preceding or following embodiment/feature/aspect, wherein the high molecular weight polyester polymer is poly(3-hydroxybutyrate-co-4-hydroxybutyrate), poly(4-hydroxybutyrate), D-polylactic acid, L-polylactic acid, or any combination thereof.
3. The method of any preceding or following embodiment/feature/aspect, wherein the high molecular weight polyester polymer is polyhydroxybutyrate polymer or polylactic acid polymer or both, and the hydroxyl-terminated reaction product is hydroxyl-terminated polyhydroxybutyrate polymer, hydroxyl-terminated polylactic acid, or both.
4. The method of any preceding or following embodiment/feature/aspect, wherein the reacting step comprising hydrolytic chain scission reactions.
5. The method of any preceding or following embodiment/feature/aspect, wherein the reacting of the molten high molecular weight polyester polymer with the difunctional hydroxylated insertion compound is conducted in the presence of transesterification catalyst.
6. The method of any preceding or following embodiment/feature/aspect, further comprising separating and removing said catalyst from the hydroxyl-terminated reaction product.
7. The method of any preceding or following embodiment/feature/aspect, wherein the difunctional hydroxylated insertion compound is 1,2-propanediol, 1,3-propanediol, 1,2-butanediol, 1,3-butanediol, 1,4-butanediol, trimethylolpropane, diethanolamine, 2-amino-2-methyl propanol, glycerol, dipropylene glycol, or sorbitol, or any combination thereof.
8. The method of any preceding or following embodiment/feature/aspect, wherein the high molecular weight polyester polymer comprises poly(3-hydroxybutyrate-co-4-hydroxybutyrate) and the difunctional hydroxylated insertion compound comprises primary hydroxyl group or at least one secondary hydroxyl group, or both.
9. The method of any preceding or following embodiment/feature/aspect, wherein the high molecular weight polyester polymer comprises poly(4-hydroxybutyrate) and the difunctional hydroxylated insertion compound comprises primary hydroxyl groups.
10. The method of any preceding or following embodiment/feature/aspect, wherein molten polyester polymer having a weight average molecular weight (M_{w}) of at least about 225,000, and the hydroxyl-terminated reaction product having a weight average molecular weight (M_{w}) of from about 200,000 to about 250.
11. The method of any preceding or following embodiment/feature/aspect, wherein the melting of the high molecular weight polyester polymer comprises heating a solid form of polyester polymer to a temperature and for a time sufficient to provide a flowable molten form of the polyester polymer in which the difunctional hydroxylated insertion compound is substantially uniformly distributable.
12. The method of any preceding or following embodiment/feature/aspect, wherein the melting of the high molecular weight polyester polymer comprises heating a solid form of the polyester polymer to a temperature sufficient to provide a flowable molten form of the polyester polymer, and the reacting comprises heating the polymer and insertion compound in contact with each other at a temperature of from about 150° C. to about 190° C. for from about 2 minutes to about 120 minutes.
13. The method of any preceding or following embodiment/feature/aspect, wherein the reacting is carried out using a twin screw extruder, brabender mixer, or short mixing extruder directly into a heated stirred tank with inert gas blanket.
14. The method of any preceding or following embodiment/feature/aspect, wherein the reacting comprising selectively cleaving ester groups in the polyhydroxyalkanoate polymer to produce a hydroalkanoate ester on one side of the polymer chain and a hydroxyl functional group attached to the alkanoate acid group on the other side of the polymer chain.
15. The method of any preceding or following embodiment/feature/aspect, wherein the linkage between the difunctional hydroxylated insertion group and the terminal hydroxyacid repeat unit is more stable to further insertion or transesterification chemistry than the majority of ester groups in the polyhydroxyalkanote polymer and is resistant to replacement during the depolymerization process.
16. The method of any preceding or following embodiment/feature/aspect, wherein the difunctional hydroxylated insertion compound has a molecular weight less than 250 and has at least one primary hydroxyl group or amine group designed to insert into the polymer chain and at least one additional hydroxyl group have the same or different functionality to the hydroxyl group on the alkanoate repeat unit.
17. The method of any preceding or following embodiment/feature/aspect, wherein difunctional hydroxylated insertion compound is an aminoalcohol having either a primary or secondary amine functionality and at least one primary or secondary hydroxyl group.
18. The method of any preceding or following embodiment/feature/aspect, wherein the difunctional hydroxylated insertion compound is an aminoalcohol that is ethanolamine, diethanolamine, amino-1-propanol, amino-2-propanol, or 2-amino-2-methylpropanol.
19. The method of any preceding or following embodiment/feature/aspect, wherein the difunctional hydroxylated insertion compound is a polyhydroxyalcohol having at least one primary alcohol.
20. The method of any preceding or following embodiment/feature/aspect, wherein the difunctional hydroxylated insertion compound is a polyhydroxyalcohol that is glycerol, propylene glycol, dipropylene glycol, or sorbitol, or any combinations thereof.
21. A method of producing biobased hydroxyl-terminated polyester comprising: dispersing high molecular weight polyester polymer in liquid hydrocarbonaceous medium, wherein the polyester polymer comprising polyhydroxyalkanoate polymer, to provide dispersed polyester polymer; reacting the dispersed polyester polymer with difunctional hydroxylated insertion compound at sufficient temperature and for sufficient time to depolymerize the dispersed polyester polymer to provide at least one of hydroxyl-terminated reaction product having reduced molecular weight as compared to that of the dispersed polyester polymer, wherein the difunctional hydroxylated insertion compound is diol, aminoalcohol, or polyhydroxyalcohol, or any combination thereof, and the hydroxyl-terminated reaction product comprises hydroxyl-terminated polyhydroxyalkanoate.
22. The method of any preceding or following embodiment/feature/aspect, wherein the liquid hydrocarbonaceous medium comprises liquid hydrocarbonaceous solvent which dissolves the polyester polymer and is substantially inert with respect to the reacting.
23. The method of any preceding or following embodiment/feature/aspect, wherein the liquid hydrocarbonaceous solvent is chloroform, dichloromethane, dioxane, tetrahydrofuran, dimethyl formamide, dimethyl sulfoxide, or any combinations thereof.
24. The method of any preceding or following embodiment/feature/aspect, wherein the liquid hydrocarbonaceous medium comprises a heat transfer fluid.
25. The method of any preceding or following embodiment/feature/aspect, wherein the heat transfer fluid is toluene, white mineral oil, or any combinations thereof.
26. The method of any preceding or following embodiment/feature/aspect, wherein the liquid hydrocarbonaceous medium is butanol, and the difunctional hydroxylated insertion compound is aminoalcohol, wherein the combination of butanol and aminoalcohol is effective to increase solubility of reaction mix and increase conversion rate of the polyhydroxyalkanoate to hydroxyl-terminated polyhydroxyalkanoate.
27. The method of any preceding or following embodiment/feature/aspect, wherein the polyester polymer is polylactic acid.
28. The method of any preceding or following embodiment/feature/aspect, wherein the high molecular weight polyester polymer is poly(3-hydroxybutyrate-co-4-hydroxybutyrate), poly(4-hydroxybutyrate), D-polylactic acid, L-polylactic acid, or any combination thereof.
29. The method of any preceding or following embodiment/feature/aspect, wherein the high molecular weight polyester polymer is polyhydroxybutyrate polymer or polylactic acid polymer, or both, and the hydroxyl-terminated reaction product is hydroxyl-terminated polyhydroxybutyrate polymer or hydroxyl-terminated polylactic acid, or both.
30. The method of any preceding or following embodiment/feature/aspect, wherein the reacting step comprising hydrolytic chain scission reactions.
31. The method of any preceding or following embodiment/feature/aspect, wherein the reacting of the dispersed polyester polymer with the difunctional hydroxylated insertion compound is conducted in the presence of transesterification catalyst.
32. The method of any preceding or following embodiment/feature/aspect, further comprising separating and removing said catalyst from the hydroxyl-terminated reaction product.
33. The method of any preceding or following embodiment/feature/aspect, wherein the difunctional hydroxylated insertion compound is 1,2-propanediol, 1,3-propanediol, 1,2-butanediol, 1,3-butanediol, 1,4-butanediol, trimethylolpropane, diethanolamine, 2-amino-2-methyl propanol, glycerol, dipropylene glycol, or sorbitol, or any combination thereof.
34. The method of any preceding or following embodiment/feature/aspect, wherein the high molecular weight polyester polymer comprises poly(3-hydroxybutyrate-co-4-hydroxybutyrate) and the difunctional hydroxylated insertion compound comprises primary hydroxyl group or at least one secondary hydroxyl group, or both
35. The method of any preceding or following embodiment/feature/aspect, wherein the high molecular weight polyester polymer comprises poly(4-hydroxybutyrate) and the difunctional hydroxylated insertion compound comprises primary hydroxyl groups.
36. The method of any preceding or following embodiment/feature/aspect, wherein dispersed polyester polymer having a weight average molecular weight (M_{w}) of at least about 225,000, and the hydroxyl-terminated reaction product having a weight average molecular weight (M_{w}) of from about 200,000 to about 250.
37. The method of any preceding or following embodiment/feature/aspect, wherein the reacting comprising selectively cleaving ester groups in the polyhydroxyalkanoate polymer to produce a hydroalkanoate ester on one side of the polymer chain and a hydroxyl functional group attached to the alkanoate acid group on the other side of the polymer chain.
38. The method of any preceding or following embodiment/feature/aspect, wherein the linkage between the difunctional hydroxylated insertion group and the terminal hydroxyacid repeat unit is more stable to further insertion or transesterification chemistry than the majority of ester groups in the polyhydroxyalkanote polymer and is resistant to replacement during the depolymerization process.
39. The method of any preceding or following embodiment/feature/aspect, wherein the difunctional hydroxylated insertion compound has a molecular weight less than 250 and has at least one primary hydroxyl group or amine group designed to insert into the polymer chain and at least one additional hydroxyl group have the same or different functionality to the hydroxyl group on the alkanoate repeat unit.
40. The method of any preceding or following embodiment/feature/aspect, wherein difunctional hydroxylated insertion compound is an aminoalcohol having either a primary or secondary amine functionality and at least one primary or secondary hydroxyl group.
41. The method of any preceding or following embodiment/feature/aspect, wherein the difunctional hydroxylated insertion compound is an aminoalcohol that is ethanolamine, diethanolamine, amino-1-propanol, amino-2-propanol, or 2-amino-2-methylpropanol, or any combination thereof.
42. The method of any preceding or following embodiment/feature/aspect, wherein the difunctional hydroxylated insertion compound is a polyhydroxyalcohol having at least one primary alcohol.
43. The method of any preceding or following embodiment/feature/aspect, wherein the difunctional hydroxylated insertion compound is a polyhydroxyalcohol that is glycerol, propylene glycol, dipropylene glycol, or sorbitol.
44. A method of producing a polyurethane comprising reacting the hydroxyl-terminated reaction product of any preceding or following embodiment/feature/aspect with polyfunctional isocyanate to form a polyurethane product.
45. A composition comprising the hydroxyl-terminated reaction product of any preceding or following embodiment/feature/aspect.
46. A product made with the hydroxyl-terminated reaction product of any preceding or following embodiment/feature/aspect.
47. An article comprising the polyurethane product of any preceding or following embodiment/feature/aspect.
48. Polyhydroxyalkanoate polyols having a molecular weight from about 200,000 to about 250 produced by the controlled/systematic depolymerization of high molecular weight biobased polyhydroxyalkanoate derived polymers with a difunctional hydroxylated insertion compound, wherein the molecular weight of starting polyhydroxyalkanoate is at least about 4 times greater than that of polyhydroxyalkanoate polyol reaction products of the depolymerization.

The present invention can include any combination of these various features or embodiments above and/or below as set forth in sentences and/or paragraphs. Any combination of disclosed features herein is considered part of the present invention and no limitation is intended with respect to combinable features.

The present invention will be further clarified by the following examples, which are intended to be purely exemplary of the invention.

### EXAMPLES

### Example 1

Hydroxyl-terminated polyhydroxyalkanoate is prepared from the reaction of poly(3-hydroxybutyrate-co-4-hydroxybutyrate) with diols and polyols. This example describes methods and conditions for producing biobased, hydroxyl-terminated poly(3-hydroxybutyrate-co-4-hydroxybutyrate) (P3HB-4HB) molecules where the hydroxyl groups all have equal reactivity. Bulk transesterification of poly(3-hydroxybutyrate-co-4-hydroxybutyrate copolymers with diols can be carried out by first melting the copolymer at 150-180° C. in a twin screw extruder, brabender mixer, or other short mixing extruder connected to a heated, stirred reaction vessel under nitrogen blanket. Once the polymer is in the molten state, a diol with 0.1-10% by weight predispersed transesterification catalyst is added to the extruder/mixer. The total amount of diol added is generally 1-2 times the stoichiometric amount required to produce the desired number of hydrolytic chain scissions to react with the PHA at the target molecular weight. Mixing of the PHA, diol and catalyst can be carried out anywhere from 5-90 minutes; the longer the mixing time the lower the molecular weight of the final product. Once the desired molecular weight or viscosity is reached, the final product is pumped from the extruder/mixer into a holding tank or through a decolorizing filtration unit and then into a holding tank.

The transesterification catalysts for use in this reaction can be dibutyl tin laurate (DBTL), dibutyl tin oxide (DBTO) or zinc stearate all of which are available from Sigma Aldrich. The preferred diols to use for the transesterification are those that have at least one primary and at least one secondary hydroxyl group present. This is so that the hydroxyl groups on the PHA diol will have equal reactivity towards isocyanate groups when incorporated into polyurethanes, e.g., they are all secondary hydroxyls. Some examples of these type of diols include 1,2-propanediol, 1,2-butanediol, and 1,3-butanediol. Amino alcohols such as diethanolamine or 2-amino-2-methyl propanol may also be used (all of which are available from Sigma Aldrich). The P3HB-4HB copolymers used for the transesterification reaction typically have starting molecular weights in the 450,000-700,000 range with a %4HB content of 4-33%.

A typical formulation and continuous process for making a 1,2-propanediol-terminated, 11% 4HB P3HB-4HB copolymer (M_{w}˜650,000) would include initially mixing 1% DBTL catalyst with 1,2-propanediol. Next the 11% 4HB P3HB-4HB copolymer would be melt processed with 5% of the diol/catalyst mixture in a twin screw extruder (Leistriz, counter rotating TSE extruder under the following processing conditions (feed zone to die): 170° C./170° C./170° C./170° C./170° C./170° C./170° C./170° C./170° C./170° C.). Residence time in the extruder can be adjusted by varying the screw feed rate. For a total residence time in the extruder of 5 minutes, a solid 1,2-propanediol-terminated P3HB-4HB oligomer with an M_{w}˜30,000 would be produced once cooled to room temperature. If the residence time is extended to 70 minutes, a low viscosity liquid of a 1,2-propanediol-terminated P3HB-4HB copolymer with M_{w}˜5000 would be produced.

### Example 2

An alternative route to the bulk transesterification during melt processing such as shown in Example 1 is to carry out the transesterification reaction of P3HB-4HB in a marginal solvent such as butanol. When a P3HB-4HB copolymer (M_{w}˜500,000) was suspended in butanol and heated to 93° C., it became slightly swollen over a 7 hour period. After the addition of either diethanolamine or 2-amino-2-methylpropanol (at a ratio of amino alcohol/PHA of 0.05 to 1:1), the P3HB-4HB was rapidly dispersed within 15 minutes and conversion of the PHA to a polyol occurred within 5 hours.

### Example 3

In this example, hydroxyl-terminated polyhydroxyalkanoate is prepared from the reaction of poly-4-hydroxybutyrate with diols or polyols. This example describes methods and conditions for producing biobased, hydroxyl-terminated poly-4-hydroxybutyrate (P4HB) molecules where the hydroxyl groups all have equal reactivity. Unlike the P3HB-4HB copolymers as described in Example 1, all of the terminal hydroxyls present in P4HB polymers are primary hydroxyls. Therefore in order to produce P4HB diols and polyols where all the hydroxyls groups would be primary and have equal reactivity to isocyanates for making polyurethanes, P4HB needs to be transesterified with diols or polyols which themselves contain primary hydroxyl groups. Diols and polyols that are most appropriate for reaction with P4HB therefore include 1,3-propanediol, 1,4-butanediol and trimethylolpropane (Sigma Aldrich). In addition, P4HB is more thermally stable than P3HB-4HB copolymers due to the absence of 3HB units which undergo chain scission reactions via β-elimination reactions at relatively lower temperatures. The P4HB polymer can therefore be processed at higher temperatures to speed up the rate of the transesterification reaction. Similar catalysts and catalyst loading levels as described in Example 1 can be used for the P4HB transesterification reaction. However, with diols and polyols containing primary hydroxyls, the catalyst is not needed but can optionally be added. Note also that there is the added possibility in this reaction of producing P4HB-diol-P4HB as well as P4HB-diol molecules. Either compound is appropriate for preparing polyurethane formulations as they both contain primary hydroxyl-terminated compounds.

A typical formulation and batch process for making a 1,3-propanediol-terminated, P4HB (M_{w}˜426,000) would include initially mixing 0.5% DBTL catalyst with 1,3-propanediol. Next the P4HB polymer would be premelted in a brabender mixer at 180° C. and 10% by weight of the diol/catalyst mixture added to the brabender. A residence time in the mixer of 5 minutes, produces a hard waxy solid of 1,3-propanediol-terminated P4HB oligomer once cooled to room temperature. If the residence time is extended to 15 minutes, a low viscosity liquid of a 1,3-propanediol-terminated P4HB copolymer would be produced. Note that the reaction time to produce a low molecular liquid is almost five times faster than for producing the same with a P3HB-4HB copolymer. The above experiment was repeated with 1,4-butanediol as the diol which also produced a waxy solid after 5 minutes of processing at 180° C. and a liquid after 15 minutes. Alternate catalysts to use for this reaction could include DBTO and zinc stearate.

A continuous process for forming 1,3-propanediol-terminated P4HB oligomers can be carried out by first premixing the P4HB polymer with 1% catalyst and 0.5% diol. The presence of the diol liquid helps to wet the catalyst and allows easier dispersion into the P4HB. This mixture is then compounded in a Leistriz twin screw extruder at a feed rate of ˜1 kg/hr. The temperature profile of the extruder would be (feed zone to die): 170° C./170° C./170° C./170° C./170° C./170° C./170° C./170° C./170° C./170° C. The initial screw speed would be 25 rpm. The remaining 1,3-propandiol is pumped into the melt 2L/D section of the extruder from the initial feed port such that the ratio of P4HB/diol is 90/10. Total residence time under these conditions is ˜10 minutes which is sufficient to continuously producing a waxy solid after to cooling to room temperature. Slowing the screw speed to 10 rpm would increase the residence time of the reactants to 20-25 minutes thereby producing a liquid P4HB-1,3-propanediol material.

The reaction described above could also be carried out in the presence of a solvent (chloroform, toluene, dioxane, THF, dimethyl formamide and dimethyl sulfoxide), heat transfer fluid (mineral oil-type), or plasticizers. The advantage of using a solvent is the ability to carry out the transesterification reaction at much lower temperatures thereby minimizing thermal break down of the PHA polymers. Heat transfer fluids are also helpful to use during extrusion and conversion of the PHA especially at the higher temperatures in order to minimize side reaction products which may cause problems with the final PHA-diol or -polyol depending on the application.

### Example 4

In this example, hydroxyl-terminated polylactic acid (PLA) is prepared from the reaction of PLA with diols or polyols. Otherwise following the procedures of Examples of 1 and 2, the P3HB-4HB or P4HB polymer can be replaced by another biobased polymer polylactic acid (D or L form or any combination of the two) to produce hydroxyl-terminated polylactic acid compounds. The advantages of using PLA to produce biobased diols and polyols is that it is more thermally stable than PHB and can be processed at even higher temperatures than PHB (150-190° C.) and therefore at faster rates. Also it produces polyester diols or polyols with a different range of properties for production of polyurethanes. PLA is soluble in a variety of solvents and therefore using the solution transesterification reaction method, can be prepared under a wider processing window.

### Example 5

Biobased polyurethanes are prepared using any of the biobased PHA or PLA diols and polyols described in Examples 1-4. The biobased PHA or PLA diols and polyols described in Examples 1-4 are readily converted into biobased polyurethane products through reaction with difunctional or polyfunctional isocyanates such as TDI, MDI, pMDI, IPDI, HMDI, di-isocyanato butane or lysine diisocyanate. Catalysts such as DBTL may also be added to aid in the reaction of PHA or PLA-hydroxyls with the isocyanate groups.

Applicant(s) specifically incorporates the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method of producing a biobased hydroxyl-terminated polyester, comprising:
melting a high molecular weight polyester polymer comprising polyhydroxyalkanoate polymer, to provide molten polyester polymer;
reacting the molten polyester polymer with difunctional hydroxylated insertion compound at sufficient temperature and for sufficient time to depolymerize the molten polyester polymer to provide at least one of hydroxyl-terminated reaction product having reduced molecular weight as compared to that of the molten polyester polymer, wherein the difunctional hydroxylated insertion compound is diol, aminoalcohol, or polyhydroxyalcohol, or any combination thereof, and the hydroxyl-terminated reaction product comprises hydroxyl-terminated polyhydroxyalkanoate.

2. The method of claim 1, wherein the high molecular weight polyester polymer is poly(3-hydroxybutyrate-co-4-hydroxybutyrate), poly(4-hydroxybutyrate), D-polylactic acid, L-polylactic acid, or any combination thereof.

3. The method of claim 1, wherein the high molecular weight polyester polymer is polyhydroxybutyrate polymer or polylactic acid polymer or both, and the hydroxyl-terminated reaction product is hydroxyl-terminated polyhydroxybutyrate polymer, hydroxyl-terminated polylactic acid, or both.

4. The method of claim 1, wherein the reacting step comprising hydrolytic chain scission reactions.

5. The method of claim 1, wherein the reacting of the molten high molecular weight polyester polymer with the difunctional hydroxylated insertion compound is conducted in the presence of transesterification catalyst.

6. The method of claim 5, further comprising separating and removing said catalyst from the hydroxyl-terminated reaction product.

7. The method of claim 1, wherein the difunctional hydroxylated insertion compound is 1,2-propanediol, 1,3-propanediol, 1,2-butanediol, 1,3-butanediol, 1,4-butanediol, trimethylolpropane, diethanolamine, 2-amino-2-methyl propanol, glycerol, dipropylene glycol, or sorbitol, or any combination thereof.

8. The method of claim 1, wherein the high molecular weight polyester polymer comprises poly(3-hydroxybutyrate-co-4-hydroxybutyrate) and the difunctional hydroxylated insertion compound comprises primary hydroxyl group or at least one secondary hydroxyl group, or both.

9. The method of claim 1, wherein the high molecular weight polyester polymer comprises poly(4-hydroxybutyrate) and the difunctional hydroxylated insertion compound comprises primary hydroxyl groups.

10. The method of claim 1, wherein molten polyester polymer having a weight average molecular weight (M_{w}) of at least about 225,000, and the hydroxyl-terminated reaction product having a weight average molecular weight (M_{w}) of from about 200,000 to about 250.

11. The method of claim 1, wherein the melting of the high molecular weight polyester polymer comprises heating a solid form of polyester polymer to a temperature and for a time sufficient to provide a flowable molten form of the polyester polymer in which the difunctional hydroxylated insertion compound is substantially uniformly distributable.

12. The method of claim 1, wherein the melting of the high molecular weight polyester polymer comprises heating a solid form of the polyester polymer to a temperature sufficient to provide a flowable molten form of the polyester polymer, and the reacting comprises heating the polymer and insertion compound in contact with each other at a temperature of from about 150° C. to about 190° C. for from about 2 minutes to about 120 minutes.

13. The method of claim 1, wherein the reacting is carried out using a twin screw extruder, brabender mixer, or short mixing extruder directly into a heated stirred tank with inert gas blanket.

14. The method of claim 1, wherein the reacting comprising selectively cleaving ester groups in the polyhydroxyalkanoate polymer to produce a hydroalkanoate ester on one side of the polymer chain and a hydroxyl functional group attached to the alkanoate acid group on the other side of the polymer chain.

15. The method of claim 1, wherein the linkage between the difunctional hydroxylated insertion group and the terminal hydroxyacid repeat unit is more stable to further insertion or transesterification chemistry than the majority of ester groups in the polyhydroxyalkanote polymer and is resistant to replacement during the depolymerization process.

16. The method of claim 1, wherein the difunctional hydroxylated insertion compound has a molecular weight less than 250 and has at least one primary hydroxyl group or amine group designed to insert into the polymer chain and at least one additional hydroxyl group have the same or different functionality to the hydroxyl group on the alkanoate repeat unit.

17. The method of claim 1, wherein difunctional hydroxylated insertion compound is an aminoalcohol having either a primary or secondary amine functionality and at least one primary or secondary hydroxyl group.

18. The method of claim 1, wherein the difunctional hydroxylated insertion compound is an aminoalcohol that is ethanolamine, diethanolamine, amino-1-propanol, amino-2-propanol, or 2-amino-2-methylpropanol.

19. The method of claim 1, wherein the difunctional hydroxylated insertion compound is a polyhydroxyalcohol having at least one primary alcohol.

20. The method of claim 1, wherein the difunctional hydroxylated insertion compound is a polyhydroxyalcohol that is glycerol, propylene glycol, dipropylene glycol, or sorbitol, or any combinations thereof.

21. A method of producing biobased hydroxyl-terminated polyester comprising:
dispersing high molecular weight polyester polymer in liquid hydrocarbonaceous medium, wherein the polyester polymer comprising polyhydroxyalkanoate polymer, to provide dispersed polyester polymer;
reacting the dispersed polyester polymer with difunctional hydroxylated insertion compound at sufficient temperature and for sufficient time to depolymerize the dispersed polyester polymer to provide at least one of hydroxyl-terminated reaction product having reduced molecular weight as compared to that of the dispersed polyester polymer, wherein the difunctional hydroxylated insertion compound is diol, aminoalcohol, or polyhydroxyalcohol, or any combination thereof, and the hydroxyl-terminated reaction product comprises hydroxyl-terminated polyhydroxyalkanoate.

22. The method of claim 21, wherein the liquid hydrocarbonaceous medium comprises liquid hydrocarbonaceous solvent which dissolves the polyester polymer and is substantially inert with respect to the reacting.

23. The method of claim 21, wherein the liquid hydrocarbonaceous solvent is chloroform, dichloromethane, dioxane, tetrahydrofuran, dimethyl formamide, dimethyl sulfoxide, or any combinations thereof.

24. The method of claim 21, wherein the liquid hydrocarbonaceous medium comprises a heat transfer fluid.

25. The method of claim 24, wherein the heat transfer fluid is toluene, white mineral oil, or any combinations thereof.

26. The method of claim 21, wherein the liquid hydrocarbonaceous medium is butanol, and the difunctional hydroxylated insertion compound is aminoalcohol, wherein the combination of butanol and aminoalcohol is effective to increase solubility of reaction mix and increase conversion rate of the polyhydroxyalkanoate to hydroxyl-terminated polyhydroxyalkanoate.

27. The method of claim 26, wherein the polyester polymer is polylactic acid.

28. The method of claim 21, wherein the high molecular weight polyester polymer is poly(3-hydroxybutyrate-co-4-hydroxybutyrate), poly(4-hydroxybutyrate), D-polylactic acid, L-polylactic acid, or any combination thereof.

29. The method of claim 21, wherein the high molecular weight polyester polymer is polyhydroxybutyrate polymer or polylactic acid polymer, or both, and the hydroxyl-terminated reaction product is hydroxyl-terminated polyhydroxybutyrate polymer or hydroxyl-terminated polylactic acid, or both.

30. The method of claim 21, wherein the reacting step comprising hydrolytic chain scission reactions.

31. The method of claim 21, wherein the reacting of the dispersed polyester polymer with the difunctional hydroxylated insertion compound is conducted in the presence of transesterification catalyst.

32. The method of claim 31, further comprising separating and removing said catalyst from the hydroxyl-terminated reaction product.

33. The method of claim 21, wherein the difunctional hydroxylated insertion compound is 1,2-propanediol, 1,3-propanediol, 1,2-butanediol, 1,3-butanediol, 1,4-butanediol, trimethylolpropane, diethanolamine, 2-amino-2-methyl propanol, glycerol, dipropylene glycol, or sorbitol, or any combination thereof.

34. The method of claim 21, wherein the high molecular weight polyester polymer comprises poly(3-hydroxybutyrate-co-4-hydroxybutyrate) and the difunctional hydroxylated insertion compound comprises primary hydroxyl group or at least one secondary hydroxyl group, or both

35. The method of claim 21, wherein the high molecular weight polyester polymer comprises poly(4-hydroxybutyrate) and the difunctional hydroxylated insertion compound comprises primary hydroxyl groups.

36. The method of claim 21, wherein dispersed polyester polymer having a weight average molecular weight (M_{w}) of at least about 225,000, and the hydroxyl-terminated reaction product having a weight average molecular weight (M_{w}) of from about 200,000 to about 250.

37. The method of claim 21, wherein the reacting comprising selectively cleaving ester groups in the polyhydroxyalkanoate polymer to produce a hydroalkanoate ester on one side of the polymer chain and a hydroxyl functional group attached to the alkanoate acid group on the other side of the polymer chain.

38. The method of claim 21, wherein the linkage between the difunctional hydroxylated insertion group and the terminal hydroxyacid repeat unit is more stable to further insertion or transesterification chemistry than the majority of ester groups in the polyhydroxyalkanote polymer and is resistant to replacement during the depolymerization process.

39. The method of claim 21, wherein the difunctional hydroxylated insertion compound has a molecular weight less than 250 and has at least one primary hydroxyl group or amine group designed to insert into the polymer chain and at least one additional hydroxyl group have the same or different functionality to the hydroxyl group on the alkanoate repeat unit.

40. The method of claim 21, wherein difunctional hydroxylated insertion compound is an aminoalcohol having either a primary or secondary amine functionality and at least one primary or secondary hydroxyl group.

41. The method of claim 21, wherein the difunctional hydroxylated insertion compound is an aminoalcohol that is ethanolamine, diethanolamine, amino-1-propanol, amino-2-propanol, or 2-amino-2-methylpropanol, or any combination thereof.

42. The method of claim 21, wherein the difunctional hydroxylated insertion compound is a polyhydroxyalcohol having at least one primary alcohol.

43. The method of claim 21, wherein the difunctional hydroxylated insertion compound is a polyhydroxyalcohol that is glycerol, propylene glycol, dipropylene glycol, or sorbitol.

44. A method of producing a polyurethane comprising reacting the hydroxyl-terminated reaction product of claim 1 with polyfunctional isocyanate to form a polyurethane product.

45. A composition comprising the hydroxyl-terminated reaction product of claim 1.

46. A product made with the hydroxyl-terminated reaction product of claim 1.

47. An article comprising the polyurethane product of claim 44.

48. Polyhydroxyalkanoate polyols having a molecular weight from about 200,000 to about 250 produced by the controlled/systematic depolymerization of high molecular weight biobased polyhydroxyalkanoate derived polymers with a difunctional hydroxylated insertion compound, wherein the molecular weight of starting polyhydroxyalkanoate is at least about 4 times greater than that of polyhydroxyalkanoate polyol reaction products of the depolymerization.
